(19) United States
(12) Patent Application Publication
(10) Pub. No.: US 2015/0087997 A1
(43) Pub. Date: Mar. 26, 2015

(54) METHOD AND DEVICE FOR DETERMINING A BREATHING MOVEMENT OF AN OBJECT UNDER EXAMINATION

(71) Applicant: SIEMENS AKTIENGESELLSCHAFT, Munchen (DE)

(72) Inventors: SULTAN HAIDER, ERLANGEN (DE); STEFAN POPESCU, ERLANGEN (DE)

(21) Appl. No.: 14/489,854

(22) Filed: Sep. 18, 2014

(30) Foreign Application Priority Data
Sep. 25, 2013 (DE) 102013219232.0

(51) Int. Cl. A61B 5/113; A61B 5/01; A61B 5/00

(52) U.S. Cl. CPC A61B 5/1135; A61B 5/0077; A61B 5/015
USPC 600/474; 600/476

(57) ABSTRACT

A method and device for determining a breathing movement of an object under examination is provided for the method includes determining a breathing movement of an object under examination and includes receiving a mathematical breathing model, the mathematical breathing model including a displacement of a thoracic cage of the object under examination over time, using a projection means to project a structured image pattern onto a sagittal plane and onto a thoracic region of the object under examination, using a camera to record a sequence of at least two images of the thoracic region of the object under examination, and adapting the mathematical breathing model at least in dependence on the recorded sequence of images of the thoracic region of the object under examination. The invention also describes a corresponding device for determining a breathing movement of an object under examination.

### CROSS-REFERENCE TO RELATED APPLICATIONS

This application claims priority to DE Application No. 102013219232.0, having a filing date of Sep. 25, 2013, the entire contents of which are hereby incorporated by reference.

### FIELD OF TECHNOLOGY

The following relates to a method for determining a breathing movement of an object under examination. The following also relates to a corresponding device for determining a breathing movement of an object under examination.

### BACKGROUND

Examinations of patients and surgical interventions are frequently supported by imaging systems, such as X-ray units, computed tomography devices or magnetic resonance tomography devices. In such cases, there is frequently a requirement to record one or more images at a specific time during a respiratory cycle. One reason for this is that, in a state at the end of an expiration or inhalation, the thoracic cage is virtually motionless for a short period of time so that a plurality of images can be taken, for example from different perspectives, without the thoracic cage with the organs connected thereto executing any large movements between recordings. Movements during the acquisition of a plurality of images would, for example, have the result that the reconstruction of the images to form a three-dimensional image could only be performed imprecisely or would even be impossible. Monitoring or determining a breathing movement of an object under examination and waiting for a motionless period enables these so-called motion artifacts to be reduced or avoided. It is also possible to use a specific breathing pattern to draw conclusions regarding the general condition of a patient. Increasingly accelerated respiration, could for example, be indicative that the patient is at risk of a panic attack which could be avoided by aborting an examination.

One known possibility for determining a breathing movement of an object under examination consists in using a chest belt and an acceleration sensor in order to detect the raising and lowering of the thoracic cage. One of the drawbacks of this method is that the patient has to be fitted with the chest belt and, in many cases, the chest belt is also subject to sterility requirements.

### SUMMARY

An aspect relates to a device for determining a breathing movement of an object under examination which is easier to handle than known solutions. A further aspect relates to a corresponding method.

Another aspect relates to a method for determining a breathing movement of an object under examination and a device for determining a breathing movement of an object under examination.

Embodiments of the method for determining a breathing movement of an object under examination may comprise the following method steps:

S1) the reception of a mathematical breathing model, said mathematical breathing model comprising a displacement of a thoracic cage of the object under examination over time;

S2) the use of projection means to project a structured image pattern onto to a sagittal plane and onto a thoracic region of the object under examination;

S3) the use of a camera to record a sequence of at least two images of the thoracic region of the object under examination;

S4) the adaptation of the mathematical breathing model at least in dependence on the recorded sequence of images of the thoracic region of the object under examination.

Embodiments of the invention include determining a breathing movement of an object under examination, such as a human patient. In the first method step, a mathematical breathing model may be received, loaded or obtained. The mathematical breathing model can be used to describe a displacement of a thoracic cage of the object under examination over time. Mathematical models are known per se. They can, for example, be obtained empirically or by physical modeling. Mathematical models generally comprise parameters to be determined, a priori determined constants and mathematical linking of the parameters and constants. The determination of the parameters enables the mathematical breathing model to be adapted to the real individual object under examination. For example, the mathematical breathing model enables a prognosis of the temporal course of the displacement of the thoracic cage. This then makes is possible to wait for the time at which the thoracic cage is motionless in order to then take one or more images. A simple breathing model could, for example, comprise a sine function with which frequency and amplitude are defined as parameters to be determined.

In the second method step, a projection means, for example a projector, transmitted-light projector or a laser projector which is known per se, can be used to project a structured image pattern onto to a sagittal plane and onto a thoracic region of the object under examination. The structured image pattern can be projected onto the lateral thoracic region of the object under examination since that is where the greatest displacement during respiration occurs. A structured image pattern can be understood to mean a sequence of differently-colored patterns or patterns with different brightnesses. There may be a high contrast between the different structures, for example as with a black-and-white pattern, and the structural width of the structured image pattern can be prespecified. For example, a sequence of different color and/or brightness may range from one millimeter to one centimeter. Structured image patterns may be, for example, patterns of concentric circles, a point cloud or wave patterns.

In the third method step, a camera may be used to record a sequence of at least two images of the thoracic region of the object under examination. The images can change in dependence on the displacement of the thoracic cage since, on a large displacement of the thoracic cage, i.e. when the object under examination has inhaled, the projected, structured image pattern covers a wide region of the thoracic cage, while, on a small displacement, i.e. on exhalation, less of the thoracic cage is affected by the projection.

In the fourth method step, the mathematical breathing model may be adapted at least in dependence on the recorded sequence of images of the thoracic region of the object under examination. As described above, the content of the images changes in dependence on the displacement of the thoracic cage. The sequence of the at least two images can be sent for image processing, which, for example, using a correlation method, determines the size of a change between the images in the sequence. This information can be used to adapt or improve the mathematical breathing model. The method can be performed repeatedly and the mathematical breathing model successively adapted.

The structured image pattern may be a line pattern with parallel lines and with a pre-specifiable line spacing and a prespecifiable line width.

A line pattern with parallel lines, which may be aligned perpendicularly to the displacement movement of the thoracic cage during a breathing movement, provides a large change in images recorded during a breathing movement. The line spacing and/or the line width can for example be from one millimeter up to one centimeter. Optimal line spacing and optimal line width can, for example, be determined using a test series. The two values may also depend, for example, on the resolution of the camera used.

In an exemplary embodiment, the camera and the projection means are aligned at least approximately identically. This feature may ensure that the projection for a recording using the camera is projected optimally onto the thoracic region of the object under examination and changes induced by the breathing movement are effectively acquired by the camera.

In an exemplary embodiment, an adjustable camera is used to record a thoracic image encompassing the thoracic region of the object under examination and the thoracic image is inserted in the projection of the structured image pattern onto the thoracic region of the object under examination such that the projection area, and/or a parameter characterizing the structured image pattern, is adjusted in a prespecifiable way.

An image that contains at least the thoracic region of the object under examination can be used to align the projection means such that the thoracic region is effectively i.e. completely, covered by the projected, structured image pattern, and the projection means can be adjusted such that one or more parameters that influence the structured image pattern can be adjusted. Parameters that influence the structured image pattern are, for example the line spacing and/or the line width of a line pattern, the diameter of a circular pattern or the variance of a point cloud. Either the adjustment can be performed once by means of an image analysis of the thoracic image or a plurality of thoracic images is obtained and the parameters optimized in a closed-loop control circuit according to each thoracic image. The adjustable camera can also be identical to the camera for recording the thoracic region of the object under examination.

In an exemplary embodiment, the at least one thoracic image encompassing the thoracic region comprises depth information. If the adjustable camera supplies an image encompassing at least the thoracic region of the object under examination and containing depth information, the projection means can be aligned in one step such that the thoracic region is effectively covered by the projected, structured image pattern and the projection means can be adjusted such that one or more parameters that influence the structured image pattern can be adjusted. This can avoid iteration steps that may have been necessary in the case of a thoracic image without depth information.

Expediently, the adjustable camera may be a time-of-flight camera, a stereo camera or a triangulation system.

Time-of-flight cameras, stereo cameras or triangulation systems are means that are known per se for obtaining an image with depth information.

In an alternative embodiment of the invention, before method step S4, additionally, a thermography camera can be used to record a sequence of at least two thermography images of a nasal region of the object under examination and the thermography images are used to determine the temporal change in the temperature in the region of at least one nostril and a temperature drop in the region of the at least one nostril is assigned to an enlargement of the displacement of the thoracic cage of the object under examination and, in method step S4, the mathematical breathing model may also be adapted in dependence on the assigned change in the displacement of the thoracic cage of the object under examination.

This supplement to the method improves the adaptation of the mathematical breathing model in that further information is taken into account with respect to the breathing of the object under examination. This can be done using a sequence of thermography or thermal images to encompass the nasal region of the object under examination. Here, use is made of the effect that, on exhaling, heated air flows out of the nose which is visible in the thermography images. An image processing method detects the temperature change and converts it into a change in the displacement of the thoracic cage. Vice versa, a temperature drop in the region of the nostril is assigned to an enlargement of the displacement of the thoracic cage of the object under examination. The mathematical breathing model is then also adapted in dependence on the assigned change in the displacement of the thoracic cage of the object under examination. In one exemplary embodiment, the additional adaptation consists of determining the average from the displacement of the thoracic cage originating from the sequence of images of the thoracic region of the object under examination and from the assigned displacement of the thoracic cage of the object under examination obtained by means of the thermography images.

A further embodiment provides that an adjustable camera records a nasal image encompassing the nasal region of the object under examination and the thermography camera is aligned on the nasal region of the object under examination by means of the nasal image.

The above-described adjustable camera, which can also be identical to the camera for recording the thoracic region of the object under examination, can be used to obtain an image of the object under examination with the nasal region, called a nasal image. This can be used to align the thermography camera on the nasal region, which makes a temperature change in the thermography images more visible since more image points are able to detect a temperature change.

A further embodiment of the invention is a device for determining a breathing movement of an object under examination. The device may comprise a computing and control means, a projection means and a camera. Here,

the computing and control means can be designed to receive a mathematical breathing model, said mathematical breathing model comprising a displacement of a thoracic cage of the object under examination over time;

the projection means can be designed to project a structured image pattern onto to a sagittal plane and onto a thoracic region of the object under examination;

the camera can be designed to record a sequence of at least two images of the thoracic region of the object under examination and

the computing and control means is further designed to adapt the mathematical breathing model at least in dependence on the recorded sequence of images of the thoracic region of the object under examination.

The computing and control means, which is, for example, implemented by a computer, can, for example, by running a suitable computer program be designed to adapt the mathematical breathing model at least in dependence on the recorded sequence of images of the thoracic region of the object under examination.

In an exemplary embodiment, the device is designed to carry out one of the above-described methods.

To this end, the device, for example, comprises means, that enable it to carry out the above-described method steps.

### BRIEF DESCRIPTION

Some of the embodiments will be described in detail, with reference to the following figures, wherein like designations denote like members, wherein:

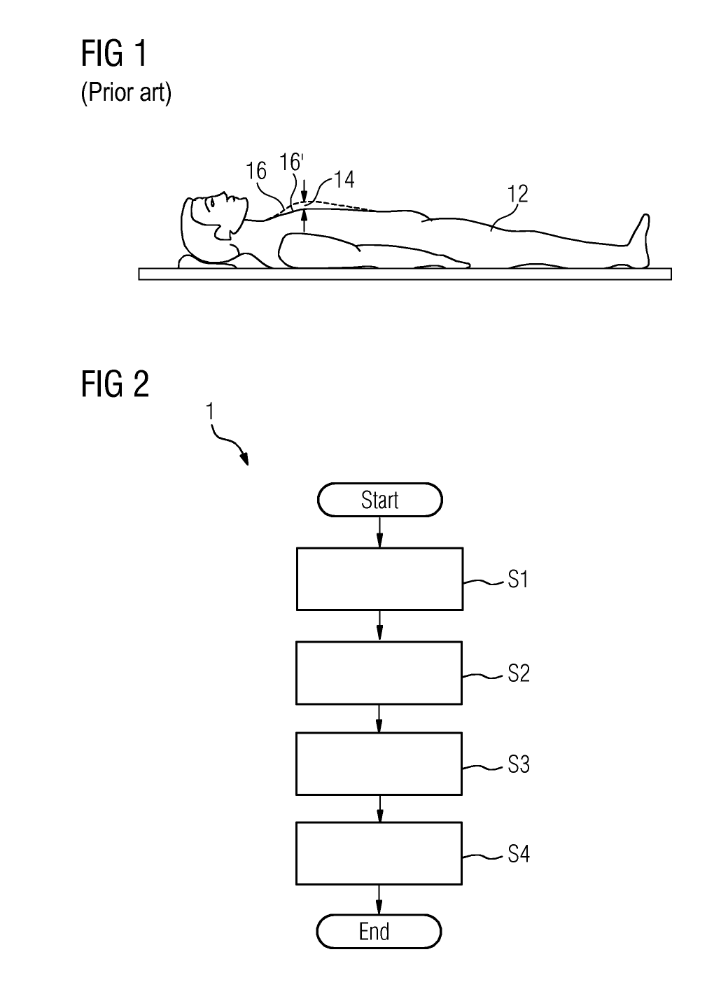

FIG. 1 depicts a schematic view of an object under examination with indicated breathing movement;

FIG. 2 depicts a flow diagram of an embodiment of a method for determining a breathing movement of an object under examination;

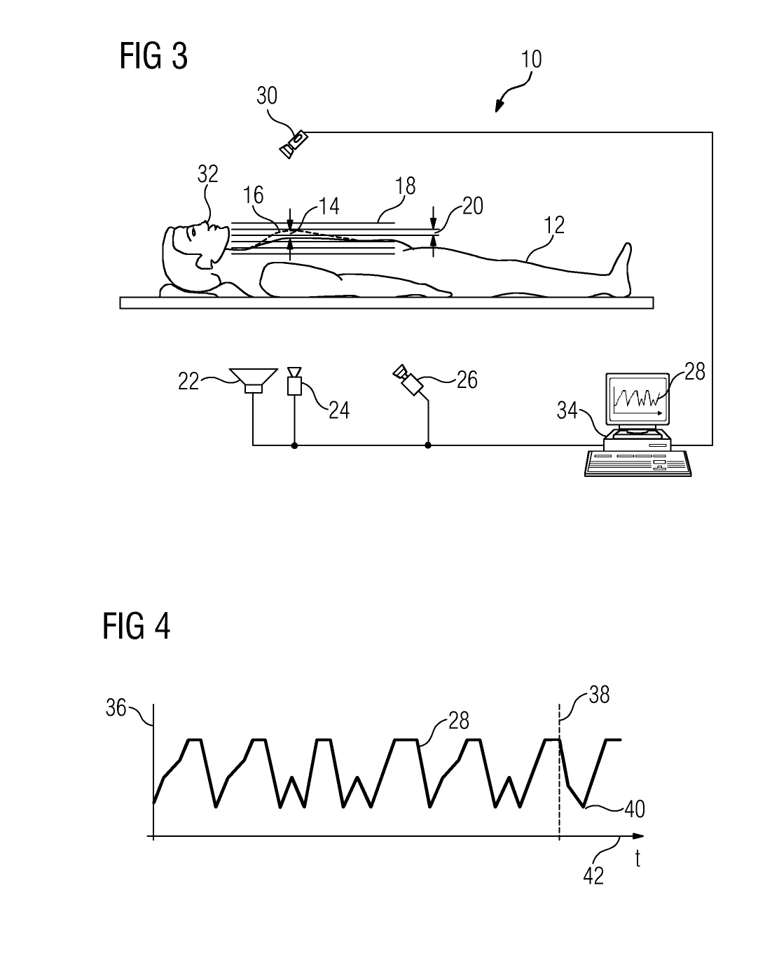

FIG. 3 depicts a schematic view of an embodiment of a device for determining a breathing movement of an object under examination; and

FIG. 4 depicts a graphical view of an example of a result of a determined breathing movement of an under examination.

### DETAILED DESCRIPTION

FIG. 1 is a depiction of an object under examination 12, here a human patient, with indicated breathing movement. The breathing movement is shown by a displacement 14 of the thoracic cage, wherein in FIG. 1, the two extreme states, namely complete inhalation and complete exhalation are depicted. The location 16 of the thoracic cage after inhalation is indicated by a dashed line, the exhalation causes the thoracic cage to move in the dorsal direction until the location 16′, which is identified by a continuous line, is reached.

FIG. 2 shows by way of example a flow diagram of a method according to the embodiments of invention 1 for determining a breathing movement of an object under examination. The method 1 may comprise the method steps S1 to S4. It starts, “Start”, with method step S1 and ends, “End”, after method step S4. The individual method steps may be as follows:

S1) reception of a mathematical breathing model, said mathematical breathing model comprising a displacement of a thoracic cage of the object under examination over time;

S2) the use of projection means to project a structured image pattern onto to a sagittal plane and onto a thoracic region of the object under examination;

S3) the use of a camera to record a sequence of at least two images of the thoracic region of the object under examination;

S4) the adaptation of the mathematical breathing model at least in dependence on the recorded sequence of images of the thoracic region of the object under examination.

The method steps may be performed at least partially automatically. Automatically performed methods are generally less error-prone and can often be performed more quickly than methods requiring manual interventions or input. It would, for example, be conceivable for a computing and control means, for example a computer, automatically to adapt the received mathematical breathing model in dependence on the recorded sequence of images of the thoracic region of the object under examination.

FIG. 3 is a symbolical depiction of an exemplary embodiment of a device 10 for determining a breathing movement of an object under examination 12. The device comprises a computing and control means 34, here a computer, a projection means 22, here a projector, and a camera 24, here a CMOS camera. The computing and control means 22 is designed to receive a mathematical breathing model in that it, for example, comprises a suitable interface for loading the mathematical breathing model into a working memory. The mathematical breathing model comprises a displacement 14 of a thoracic cage of the object under examination 12 over time. The projection means 22 is designed to project a structured image pattern 18 onto a sagittal plane and onto a thoracic region of the object under examination 12. In this exemplary embodiment, the structured image pattern 18 is a line pattern with parallel lines, which are aligned perpendicularly to the displacement movement of the thoracic cage during a breathing movement. The line spacing 20 can be prespecified and is, for example, two millimeters. The camera 24 is designed to record a sequence of a plurality of images of the thoracic region of the object under examination 12 and make it available to the computing and control means 34. The images will change in dependence on the displacement 14 of the thoracic cage since, on a large displacement 16 of the thoracic cage, i.e. when the object under examination 12 has inhaled, the projected structured image pattern 18 covers a wide region of the thoracic cage, while, on a small displacement, i.e. on exhalation, less of the thoracic cage is affected by the projection. It can be seen that the camera 24 and the projection means 22 are aligned identically. This ensures that the projection is optimally projected onto the thoracic region of the object under examination 12 for a recording by means of the camera 24 and that changes induced by the breathing movement changes are effectively acquired by the camera 24. In order to ensure that the projection of the structured image pattern 18 is as effective as possible, the device 10 comprises an adjustable camera 26, for example a so-called time-of-flight camera. An image supplied by the adjustable camera 26 enables the projection means 22 and the camera 24 to be aligned with the thoracic region of the object under examination 12. Since this exemplary embodiment entails a camera that also provides depth information, the projection means 22 and the camera 24 can be adjusted in one adjustment step to the thoracic region of the object under examination 12 since the distance of the object under examination 12 from the projection means 22 and from the camera 24 can be determined from the depth information. The device 10 in the exemplary embodiment further comprises a thermography camera 30, which is aligned with a nasal region 32 of the object under examination 12 with the aid of the adjustable camera 26. The thermography camera 30 is designed to receive a sequence of at least two thermography images of the nasal region 32 of the object under examination 12 and to make it available to the computing and control means 34. The computing and control means 34 can determine the temporal change in the temperature in the region of a nostril by means of the thermography images, for example with the aid of a image processing method, and can assign a temperature drop in the region of the nostril to an enlargement of the displacement of the thoracic cage of the object under examination 12. The computing and control means 34 is further designed to adapt the mathematical breathing model in dependence on the recorded sequence of images of the thoracic region of the object under examination 12 and in dependence on the assigned change in the displacement of the thoracic cage of the object under examination 12. The computing and control means 34 presents a graphical depiction 28 of a result of the adapted mathematical breathing model on a display means, here a monitor.

Finally, FIG. 4 is a symbolical representation of an example of a graphical depiction 28 of a determined breathing movement of an object under examination. The graphical depiction 28 represents a displacement 36 of the thoracic cage of the object under examination over time 42. Up to the point in time 38, the displacement 36 of the thoracic cage was determined by one of the above-described methods. The determination of parameters enables a mathematical breathing model to be adapted to the real individual object under examination. The mathematical breathing model facilitates, for example, a prognosis of the temporal course of the displacement 36 of the thoracic cage according to 38—this is represented by a dashed line. This then makes it possible to determine the point in time 40 at which the thoracic cage will probably be motionless, i.e. here the point in time for a predicted respiratory condition and then to wait for this point in time in order to record one or more images, which do not have any motion artifacts due to a breathing movement, by means of an imaging mechanism, such as an X-ray unit or a computed tomography device.

1. A method for determining a breathing movement of an object under examination comprising the following method steps:
S1) receiving a mathematical breathing model, the mathematical breathing model comprising a displacement of a thoracic cage of the object under examination over time;
S2) using a projection means to project a structured image pattern onto a sagittal plane and onto a thoracic region of the object under examination;
S3) using a camera to record a sequence of at least two images of the thoracic region of the object under examination; and
S4) adapting the mathematical breathing model at least in dependence on the recorded sequence of at least two images of the thoracic region of the object under examination.

2. The method as claimed in claim 1, wherein the structured image pattern is a line pattern with parallel lines and with a prespecifiable line spacing and a prespecifiable line width.

3. The method as claimed in claim 1, wherein the camera and the projection means are aligned at least approximately identically.

4. The method as claimed in claim 1, wherein an adjustable camera is used to record a thoracic image encompassing the thoracic region of the object under examination and the thoracic image is inserted into the projection of the structured image pattern onto the thoracic region of the object under examination such that the projection area, and/or a parameter characterizing the structured image pattern , is adjusted in a prespecifiable way.

5. The method as claimed in claim 4, wherein the thoracic image encompassing the thoracic region comprises depth information.

6. The method as claimed in claim 5, wherein the adjustable camera is at least one of a time-of-flight camera, a stereo camera, and a triangulation system.

7. The method as claimed in claim 1, wherein, before method step S4, additionally, a thermography camera is used to record a sequence of at least two thermography images of a nasal region of the object under examination and wherein the at least two thermography images are used to determine a temporal change in a temperature in a region of at least one nostril and wherein a temperature drop in the region of the at least one nostril is assigned to an enlargement of the displacement of the thoracic cage of the object under examination and wherein, in method step S4, the mathematical breathing model is also adapted in dependence on an assigned change in the displacement of the thoracic cage of the object under examination.

8. The method as claimed in claim 7, wherein, an adjustable camera is used to record a nasal image encompassing the nasal region of the object under examination and the thermography camera is aligned with the nasal region of the object under examination by means of the nasal image.

9. A device for determining a breathing movement of an object under examination, comprising a computing and control means, a projection means and a camera, wherein
the computing and control means is designed to receive a mathematical breathing model, the mathematical breathing model comprising a displacement of a thoracic cage of the object under examination over time;
the projection means is designed to project a structured image pattern onto a sagittal plane and onto a thoracic region of the object under examination;
the camera is designed to record a sequence of at least two images of the thoracic region of the object under examination and
the computing and control means is further designed to adapt the mathematical breathing model at least in dependence on the recorded sequence of the at least two images of the thoracic region of the object under examination.

10. A device as claimed in claim 9, wherein the device is designed to implement a method as claimed in claim 2.
